SEQUENCE LISTING (8 sequences)

SEQ ID NO: 1 - DNA, 3136 bp, Homo sapiens
  CDS (39)..(1889)
ccgctgaggccgcggcgcccgccagcctgtcccgcgccatggccccgcgcgcccggcggcgccgcccgctgttcgcgctgctgctgctctgcgcgctgctcgcccggctgcaggtggctttgcagatcgctcctccatgtaccagtgagaagcattatgagcatctgggacggtgctgtaacaaatgtgaaccaggaaagtacatgtcttctaaatgcactactacctctgacagtgtatgtctgccctgtggcccggatgaatacttggatagctggaatgaagaagataaatgcttgctgcataaagtttgtgatacaggcaaggccctggtggccgtggtcgccggcaacagcacgaccccccggcgctgcgcgtgcacggctgggtaccactggagccaggactgcgagtgctgccgccgcaacaccgagtgcgcgccgggcctgggcgcccagcacccgttgcagctcaacaaggacacagtgtgcaaaccttgccttgcaggctacttctctgatgccttttcctccacggacaaatgcagaccctggaccaactgtaccttccttggaaagagagtagaacatcatgggacagagaaatccgatgcggtttgcagttcttctctgccagctagaaaaccaccaaatgaaccccatgtttacttgcccggtttaataattctgcttctcttcgcgtctgtggccctggtggctgccatcatctttggcgtttgctataggaaaaaagggaaagcactcacagctaatttgtggcactggatcaatgaggcttgtggccgcctaagtggagataaggagtcctcaggtgacagttgtgtcagtacacacacggcaaactttggtcagcagggagcatgtgaaggtgtcttactgctgactctggaggagaagacatttccagaagatatgtgctacccagatcaaggtggtgtctgtcagggcacgtgtgtaggaggtggtccctacgcacaaggcgaagatgccaggatgctctcattggtcagcaagaccgagatagaggaagacagcttcagacagatgcccacagaagatgaatacatggacaggccctcccagcccacagaccagttactgttcctcactgagcctggaagcaaatccacacctcctttctctgaacccctggaggtgggggagaatgacagtttaagccagtgcttcacggggacacagagcacagtgggttcagaaagctgcaactgcactgagcccctgtgcaggactgattggactcccatgtcctctgaaaactacttgcaaaaagaggtggacagtggccattgcccgcactgggcagccagccccagccccaactgggcagatgtctgcacaggctgccggaaccctcctggggaggactgtgaacccctcgtgggttccccaaaacgtggacccttgccccagtgcgcctatggcatgggccttccccctgaagaagaagccagcaggacggaggccagagaccagcccgaggatggggctgatgggaggctcccaagctcagcgagggcaggtgccgggtctggaagctcccctggtggccagtcccctgcatctggaaatgtgactggaaacagtaactccacgttcatctccagcgggcaggtgatgaacttcaagggcgacatcatcgtggtctacgtcagccagacctcgcaggagggcgcggcggcggctgcggagcccatgggccgcccggtgcaggaggagaccctggcgcgccgagactccttcgcggggaacggcccgcgcttcccggacccgtgcggcggccccgaggggctgcgggagccggagaaggcctcgaggccggtgcaggagcaaggcggggccaaggcttgagcgccccccatggctgggagcccgaagctcggagccagggctcgcgagggcagcaccgcagcctctgccccagccccggccacccagggatcgatcggtacagtcgaggaagaccacccggcattctctgcccactttgccttccaggaaatgggcttttcaggaagtgaattgatgaggactgtccccatgcccacggatgctcagcagcccgccgcactggggcagatgtctcccctgccactcctcaaactcgcagcagtaatttgtggcactatgacagctatttttatgactatcctgttctgtggggggggggtctatgttttccccccatatttgtattccttttcataacttttcttgatatctttcctccctcttttttaatgtaaaggttttctcaaaaattctcctaaaggtgagggtctctttcttttctcttttccttttttttttctttttttggcaacctggctctggcccaggctagagtgcagtggtgcgattatagcccggtgcagcctctaactcctgggctcaagcaatccaagtgatcctcccacctcaaccttcggagtagctgggatcacagctgcaggccacgcccagcttcctccccccgactccccccccccagagacacggtcccaccatgttacccagcctggtctcaaactccccagctaaagcagtcctccagcctcggcctcccaaagtactgggattacaggcgtgagcccccacgctggcctgctttacgtattttcttttgtgcccctgctcacagtgttttagagatggctttcccagtgtgtgttcattgtaaacacttttgggaaagggctaaacatgtgaggcctggagatagttgctaagttgctaggaacatgtggtgggactttcatattctgaaaaatgttctatattctcatttttctaaaagaaagaaaaaaggaaacccgatttatttctcctgaatctttttaagtttgtgtcgttccttaagcagaactaagctcagtatgtgaccttacccgctaggtggttaatttatccatgctggcagaggcactcaggtacttggtaagcaaatttctaaaactccaagttgctgcagcttggcattcttcttattctagaggtctctctggaaaagatggagaaaatgaacaggacatggggctcctggaaagaaagggcccgggaagttcaaggaagaataaagttgaaattttaaaaaaaaa

SEQ ID NO: 2 - protein, 616 aa, Homo sapiens
MAPRARRRRPLFALLLLCALLARLQVALQIAPPCTSEKHYEHLGRCCNKCEPGKYMSSKCTTTSDSVCLPCGPDEYLDSWNEEDKCLLHKVCDTGKALVAVVAGNSTTPRRCACTAGYHWSQDCECCRRNTECAPGLGAQHPLQLNKDTVCKPCLAGYFSDAFSSTDKCRPWTNCTFLGKRVEHHGTEKSDAVCSSSLPARKPPNEPHVYLPGLIILLLFASVALVAAIIFGVCYRKKGKALTANLWHWINEACGRLSGDKESSGDSCVSTHTANFGQQGACEGVLLLTLEEKTFPEDMCYPDQGGVCQGTCVGGGPYAQGEDARMLSLVSKTEIEEDSFRQMPTEDEYMDRPSQPTDQLLFLTEPGSKSTPPFSEPLEVGENDSLSQCFTGTQSTVGSESCNCTEPLCRTDWTPMSSENYLQKEVDSGHCPHWAASPSPNWADVCTGCRNPPGEDCEPLVGSPKRGPLPQCAYGMGLPPEEEASRTEARDQPEDGADGRLPSSARAGAGSGSSPGGQSPASGNVTGNSNSTFISSGQVMNFKGDIIVVYVSQTSQEGAAAAAEPMGRPVQEETLARRDSFAGNGPRFPDPCGGPEGLREPEKASRPVQEQGGAKA

SEQ ID NO: 3 - protein, 232 aa, Homo sapiens
EPRSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKDYKCKVSNKALPAPMQKTISKAKGQPREPQVYTLPPSRDELTKNQVSLTCLVKGFYPRHIAVEWESNGQPENNYKTTPPVLDSDGSFFLYSKLTVDKSRWQQGNVFSCSVMHEALHNHYTQKSLSLSPGK

SEQ ID NO: 4 - DNA, 1878 bp, Murine
  CDS (1)..(1875)
atggccccgcgcgcccggcggcgccgccagctgcccgcgccgctgctggcgctctgcgtgctgctcgttccactgcaggtgactctccaggtcactcctccatgcacccaggagaggcattatgagcatctcggacggtgttgcagcagatgcgaaccaggaaagtacctgtcctctaagtgcactcctacctccgacagtgtgtgtctgccctgtggccccgatgagtacttggacacctggaatgaagaagataaatgcttgctgcataaagtctgtgatgcaggcaaggccctggtggcggtggatcctggcaaccacacggccccgcgtcgctgtgcttgcacggctggctaccactggaactcagactgcgagtgctgccgcaggaacacggagtgtgcacctggcttcggagctcagcatcccttgcagctcaacaaggatacggtgtgcacaccctgcctcctgggcttcttctcagatgtcttttcgtccacagacaaatgcaaaccttggaccaactgcaccctccttggaaagctagaagcacaccaggggacaacggaatcagatgtggtctgcagctcttccatgacactgaggagaccacccaaggaggcccaggcttacctgcccagtctcatcgttctgctcctcttcatctctgtggtagtagtggctgccatcatcttcggcgtttactacaggaagggagggaaagcgctgacagctaatttgtggaattgggtcaatgatgcttgcagtagtctaagtggaaataaggagtcctcaggggaccgttgtgctggttcccactcggcaacctccagtcagcaagaagtgtgtgaaggtatcttactaatgactcgggaggagaagatggttccagaagacggtgctggagtctgtgggcctgtgtgtgcggcaggtgggccctgggcagaagtcagagattctaggacgttcacactggtcagcgaggttgagacgcaaggagacctctcgaggaagattcccacagaggatgagtacacggaccggccctcgcagccttcgactggttcactgctcctaatccagcagggaagcaaatctatacccccattccaggagcccctggaagtgggggagaacgacagtttaagccagtgtttcaccgggactgaaagcacggtggattctgagggctgtgacttcactgagcctccgagcagaactgactctatgcccgtgtcccctgaaaagcacctgacaaaagaaatagaaggtgacagttgcctcccctgggtggtcagctccaactcaacagatggctacacaggcagtgggaacactcctggggaggaccatgaaccctttccagggtccctgaaatgtggaccattgccccagtgtgcctacagcatgggctttcccagtgaagcagcagccagcatggcagaggcgggagtacggccccaggacagggctgatgagaggggagcctcagggtccgggagctcccccagtgaccagccacctgcctctgggaacgtgactggaaacagtaactccacgttcatctctagcgggcaggtgatgaacttcaagggtgacatcatcgtggtgtatgtcagccagacctcgcaggagggcccgggttccgcagagcccgagtcggagcccgtgggccgccctgtgcaggaggagacgctggcacacagagactcctttgcgggcaccgcgccgcgcttccccgacgtctgtgccaccggggctgggctgcaggagcagggggcaccccggcagaaggacgggacatcgcggccggtgcaggagcagggtggggcgcagacttcactccatacccaggggtccggacaatgtgcagaatga

SEQ ID NO: 5 - protein, 625 aa, Murine
MAPRARRRRQLPAPLLALCVLLVPLQVTLQVTPPCTQERHYEHLGRCCSRCEPGKYLSSKCTPTSDSVCLPCGPDEYLDTWNEEDKCLLHKVCDAGKALVAVDPGNHTAPRRCACTAGYHWNSDCECCRRNTECAPGFGAQHPLQLNKDTVCTPCLLGFFSDVFSSTDKCKPWTNCTLLGKLEAHQGTTESDVVCSSSMTLRRPPKEAQAYLPSLIVLLLFISVVVVAAIIFGVYYRKGGKALTANLWNWVNDACSSLSGNKESSGDRCAGSHSATSSQQEVCEGILLMTREEKMVPEDGAGVCGPVCAAGGPWAEVRDSRTFTLVSEVETQGDLSRKIPTEDEYTDRPSQPSTGSLLLIQQGSKSIPPFQEPLEVGENDSLSQCFTGTESTVDSEGCDFTEPPSRTDSMPVSPEKHLTKEIEGDSCLPWVVSSNSTDGYTGSGNTPGEDHEPFPGSLKCGPLPQCAYSMGFPSEAAASMAEAGVRPQDRADERGASGSGSSPSDQPPASGNVTGNSNSTFISSGQVMNFKGDIIVVYVSQTSQEGPGSAEPESEPVGRPVQEETLAHRDSFAGTAPRFPDVCATGAGLQEQGAPRQKDGTSRPVQEQGGAQTSLHTQGSGQCAE

SEQ ID NO: 6 - protein, 33 aa, Artificial sequence
  Murine
RMKQIEDKIEEILSKIYHIENEIARIKKLIGER

SEQ ID NO: 7 - DNA, 954 bp, Homo sapiens
  CDS (1)..(951)
atgcgccgcgccagcagagactacaccaagtacctgcgtggctcggaggagatgggcggcggccccggagccccgcacgagggccccctgcacgccccgccgccgcctgcgccgcaccagccccccgccgcctcccgctccatgttcgtggccctcctggggctggggctgggccaggttgtctgcagcgtcgccctgttcttctatttcagagcgcagatggatcctaatagaatatcagaagatggcactcactgcatttatagaattttgagactccatgaaaatgcagattttcaagacacaactctggagagtcaagatacaaaattaatacctgattcatgtaggagaattaaacaggcctttcaaggagctgtgcaaaaggaattacaacatatcgttggatcacagcacatcagagcagagaaagcgatggtggatggctcatggttagatctggccaagaggagcaagcttgaagctcagccttttgctcatctcactattaatgccaccgacatcccatctggttcccataaagtgagtctgtcctcttggtaccatgatcggggttgggccaagatctccaacatgacttttagcaatggaaaactaatagttaatcaggatggcttttattacctgtatgccaacatttgctttcgacatcatgaaacttcaggagacctagctacagagtatcttcaactaatggtgtacgtcactaaaaccagcatcaaaatcccaagttctcataccctgatgaaaggaggaagcaccaagtattggtcagggaattctgaattccatttttattccataaacgttggtggattttttaagttacggtctggagaggaaatcagcatcgaggtctccaacccctccttactggatccggatcaggatgcaacatactttggggcttttaaagttcgagatatagattga

SEQ ID NO: 8 - protein, 317 aa, Homo sapiens
MRRASRDYTKYLRGSEEMGGGPGAPHEGPLHAPPPPAPHQPPAASRSMFVALLGLGLGQVVCSVALFFYFRAQMDPNRISEDGTHCIYRILRLHENADFQDTTLESQDTKLIPDSCRRIKQAFQGAVQKELQHIVGSQHIRAEKAMVDGSWLDLAKRSKLEAQPFAHLTINATDIPSGSHKVSLSSWYHDRGWAKISNMTFSNGKLIVNQDGFYYLYANICFRHHETSGDLATEYLQLMVYVTKTSIKIPSSHTLMKGGSTKYWSGNSEFHFYSINVGGFFKLRSGEEISIEVSNPSLLDPDQDATYFGAFKVRDID

(19) United States
(12) Patent Application Publication
(10) Pub. No.: US 2013/0101585 A1
(43) Pub. Date: Apr. 25, 2013

(54) METHOD OF INHIBITING OSTEOCLAST ACTIVITY

(71) Applicant: Immunex Corporation, Thousand Oaks, CA (US)

(72) Inventors: Dirk M. ANDERSON, Port Townsend, WA (US); Laurent J. GALIBERT, Prevessin Moens (FR)

(73) Assignee: IMMUNEX CORPORATION, Thousand Oaks, CA (US)

(21) Appl. No.: 13/717,309

(22) Filed: Dec. 17, 2012

Related U.S. Application Data
(63) Continuation of application No. 12/850,368, filed on Aug. 4, 2010, now Pat. No. 8,333,963.
Application No. 12/850,368 is a continuation of application No. 12/137,397, filed on Jun. 11, 2008, now Pat. No. 7,790,684.
Application No. 12/137,397 is a continuation of application No. 09/705,985, filed on Nov. 3, 2000, now abandoned.
Application No. 09/705,985 is a continuation of application No. PCT/US99/10588, filed on May 13, 1999.
Application No. 12/137,397 is a continuation-in-part of application No. 11/881,911, filed on Jul. 30, 2007, now Pat. No. 7,932,375.
Application No. 11/881,911 is a division of application No. 10/405,878, filed on Apr. 1, 2003, now Pat. No. 7,262,274.
Application No. 10/405,878 is a continuation of application No. 09/871,291, filed on May 30, 2001, now Pat. No. 6,562,948.
Application No. 09/871,291 is a division of application No. 09/577,800, filed on May 24, 2000, now Pat. No. 6,479,635.
Application No. 09/577,800 is a continuation of application No. 09/466,496, filed on Dec. 17, 1999, now Pat. No. 6,528,482.
Application No. 09/466,496 is a continuation of application No. 08/996,139, filed on Dec. 22, 1997, now Pat. No. 6,017,729.
(60) Provisional application No. 60/110,836, filed on Dec. 3, 1998.
(60) Provisional application No. 60/085,487, filed on May 14, 1998.
(60) Provisional application No. 60/064,671, filed on Oct. 14, 1997.
(60) Provisional application No. 60/077,181, filed on Mar. 7, 1997.
(60) Provisional application No. 60/059,978, filed on Dec. 23, 1996.

(51) Int. Cl. C07K 14/715

(52) U.S. Cl. CPC C07K 14/7151
USPC 424/134.1; 435/375; 514/16.7; 514/16.9

(57) ABSTRACT

Methods for inhibiting osteoclastogenesis by administering a soluble RANK polypeptide are disclosed. Such methods can be used to treat a variety of different cancers, including bone cancer, multiple myeloma, melanoma, breast cancer, squamous cell carcinoma, lung cancer, prostate cancer, hematologic cancers, head and neck cancer and renal cancer.

### CROSS-REFERENCE TO RELATED PATENT APPLICATIONS

This application is a continuation of U.S. patent application Ser. No. 12/850,368, filed Aug. 4, 2010, now allowed, which is incorporated herein in its entirety for all purposes, which is a continuation of U.S. patent application Ser. No. 12/137,397, filed Jun. 11, 2008, now U.S. Pat. No. 7,790,684, which is a continuation of U.S. patent application Ser. No. 09/705,985 filed Nov. 3, 2000, now abandoned, which is a continuation of International patent application No. PCT/US99/10588 filed May 13, 1999, which claims the benefit of U.S. provisional patent applications 60/110,836 filed Dec. 3, 1998 and 60/085,487 filed May 14, 1998, and is a continuation-in-part of U.S. patent application Ser. No. 11/881,911 filed Jul. 30, 2007, now U.S. Pat. No. 7,932,375 which is a divisional of U.S. patent application Ser. No. 10/405,878 filed Apr. 1, 2003, now U.S. Pat. No. 7,262,274, which is a continuation of U.S. patent application Ser. No. 09/871,291 filed May 30, 2001, now U.S. Pat. No. 6,562,948, which is a divisional of U.S. patent application Ser. No. 09/577,800 filed May 24, 2000, now U.S. Pat. No. 6,479,635, which is a continuation of U.S. patent application Ser. No. 09/466,496 filed Dec. 17, 1999, now U.S. Pat. No. 6,528,482, which is a continuation of U.S. patent application Ser. No. 08/996,139 filed Dec. 22, 1997, now U.S. Pat. No. 6,017,729, which claims the benefit of U.S. provisional application No. 60/064,671 filed Oct. 14, 1997, U.S. provisional application No. 60/077,181 filed Mar. 7, 1997, and U.S. provisional application No. 60/059,978, filed Dec. 23, 1996.

### REFERENCE TO THE SEQUENCE LISTING

The present application is being filed along with a Sequence Listing in electronic format. The Sequence Listing is provided as a file entitled 2874-US-CNT6_SEQ_ST25.txt, created Oct. 30, 2012, which is 41 KB in size. The information in the electronic format of the Sequence Listing is incorporated herein by reference in its entirety.

### TECHNICAL FIELD OF THE INVENTION

The present invention relates generally to the field of cytokine receptors, and more specifically to cytokine receptor/ligand pairs having osteoclast regulatory activity.

### BACKGROUND OF THE INVENTION

RANK (Receptor Activator of NF-κB) and its ligand (RANKL) are a recently-described receptor/ligand pair that play an important role in an immune response. The cloning of RANK and RANKL is described in U.S. Ser. No. 08/996,139 and U.S. Ser. No. 08/995,659, respectively. It has recently been found that RANKL binds to a protein referred to as osteoprotegerin (OPG), a member of the Tumor Necrosis Factor Receptor (TNFR) family. Yasuda et al. (Proc. Natl. Acad. Sci. 95:3597; 1998) expression cloned a ligand for OPG, which they referred to as osteoclastogenesis inhibitory factor. Their work was repeated by Lacey et al. (Cell 93:165; 1998). In both cases, the ligand they cloned turned out to be identical to RANKL.

In osteoclastogenesis, the interaction of an osteoblast or stromal cell with an osteoclast precursor leads to the differentiation of the precursor into an osteoclast. OPG was known to inhibit this differentiation. A model has been proposed in which RANKL on the osteoblast or stromal cell surface interacts with a specific receptor on an osteoclast progenitor surface, signaling a differentiation event. OPG effectively blocks the interaction of RANKL with a receptor on osteoclast progenitors in vitro, and has been shown to ameliorate the effects of ovariectomy on bone-loss in mice. However, OPG is also known to bind other ligands in the TNF family, which may have a deleterious effect on the activities of such ligands in vivo. Moreover, the presence of other ligands that bind OPG in vivo may require high dosages of OPG to be administered in order to have sufficient soluble OPG available to inhibit osteoclastogenesis.

Accordingly, there is a need in the art to identify soluble factors that specifically bind RANKL and inhibit the ability of RANKL to induce osteoclastogenesis without reacting with other ligands.

### SUMMARY OF THE INVENTION

The present invention provides processes associated with the use of a novel receptor, referred to as RANK (for receptor activator of NF-κB), that is a member of the TNF receptor superfamily. RANK is a Type I transmembrane protein having 616 amino acid residues, comprising an extracellular domain, transmembrane region and cytoplasmic domain. RANK interacts with various TNF Receptor Associated Factors (TRAFs); triggering of RANK results in the upregulation of the transcription factor NF-κB, a ubiquitous transcription factor that is most extensively utilized in cells of the immune system.

Soluble forms of the receptor can be prepared and used to interfere with signal transduction through membrane-bound RANK. Inhibition of RANKL-mediated signal transduction will be useful in ameliorating the effects of osteoclastogenesis and osteoclast activity in disease conditions in which there is excess bone break down. Examples of such conditions include osteoporosis, Paget's disease, cancers that may metastasize to bone and induce bone breakdown (i.e., multiple myeloma, breast cancer, some melanomas; see also Mundy, C. Cancer Suppl. 80:1546; 1997), and cancers that do not necessarily metastasize to bone, but result in hypercalcemia and bone loss (e.g. squamous cell carcinomas).

Soluble forms of RANK comprise the extracellular domain of RANK or a fragment thereof that binds RANKL. Fusion proteins of RANK may be made to allow preparation of soluble RANK. Examples of such fusion proteins include a RANK/Fc fusion protein, a fusion protein of a zipper moiety (i.e., a leucine zipper), and various tags that are known in the art. Other antagonists of the interaction of RANK and RANKL (i.e., antibodies to RANKL, small molecules) will also be useful in the inventive methods. These and other aspects of the present invention will become evident upon reference to the following detailed description of the invention.

### DETAILED DESCRIPTION OF THE INVENTION

A novel partial cDNA insert with a predicted open reading frame having some similarity to CD40 was identified and was used to hybridize to colony blots generated from a dendritic cell (DC) cDNA library containing full-length cDNAs. SEQ ID NO:1 shows the nucleotide and amino acid sequence of a predicted full-length protein.

RANK is a member of the TNF receptor superfamily; it most closely resembles CD40 in the extracellular region. RANK is expressed on epithelial cells, some B cell lines, and on activated T cells. However, its expression on activated T cells is late, about four days after activation. This time course of expression coincides with the expression of Fas, a known agent of apoptosis. RANK may act as an anti-apoptotic signal, rescuing cells that express RANK from apoptosis as CD40 is known to do. Alternatively, RANK may confirm an apoptotic signal under the appropriate circumstances, again similar to CD40. RANK and its ligand are likely to play an integral role in regulation of the immune and inflammatory response. The isolation of a DNA encoding RANK is described in U.S. Ser. No. 08/996,139, filed Dec. 22, 1997, the disclosure of which is incorporated by reference herein. U.S. Ser. No. 08/996,139 describes several forms of RANK that are useful in the present invention.

Soluble RANK comprises the signal peptide and the extracellular domain (residues 1 to 213 of SEQ ID NO:2) or a fragment thereof. Alternatively, a different signal peptide can be substituted for the native leader, beginning with residue 1 and continuing through a residue selected from the group consisting of amino acids 24 through 33 (inclusive) of SEQ ID NO:2. Other members of the TNF receptor superfamily have a region of amino acids between the transmembrane domain and the ligand binding domain that is referred to as a ‘spacer’ region, which is not necessary for ligand binding. In RANK, the amino acids between 196 and 213 are predicted to form such a spacer region. Accordingly, a soluble form of RANK that terminates with an amino acid in this region is expected to retain the ability to bind a ligand for RANK in a specific manner. Preferred C-terminal amino acids for soluble RANK peptides are selected from the group consisting of amino acids 213 and 196 of SEQ ID NO:2, although other amino acids in the spacer region may be utilized as a C-terminus. In muRANK, the amino acids between 197 and 214 are predicted to form such a spacer region. Accordingly, a soluble form of RANK that terminates with an amino acid in this region is expected to retain the ability to bind a ligand for RANK in a specific manner. Preferred C-terminal amino acids for soluble RANK peptides are selected from the group consisting of amino acids 214, and 197 of SEQ ID NO:5, although other amino acids in the spacer region may be utilized as a C-terminus. Moreover, fragments of the extracellular domain will also provide soluble forms of RANK.

Fragments can be prepared using known techniques to isolate a desired portion of the extracellular region, and can be prepared, for example, by comparing the extracellular region with those of other members of the TNFR family (of which RANK is a member) and selecting forms similar to those prepared for other family members. Alternatively, unique restriction sites or PCR techniques that are known in the art can be used to prepare numerous truncated forms which can be expressed and analyzed for activity.

Other derivatives of the RANK proteins within the scope of this invention include covalent or aggregative conjugates of the proteins or their fragments with other proteins or polypeptides, such as by synthesis in recombinant culture as N-terminal or C-terminal fusions. For example, the conjugated peptide may be a signal (or leader) polypeptide sequence at the N-terminal region of the protein which co-translationally or post-translationally directs transfer of the protein from its site of synthesis to its site of function inside or outside of the cell membrane or wall (e.g., the yeast α-factor leader).

Protein fusions can comprise peptides added to facilitate purification or identification of RANK proteins and homologs (e.g., poly-His). The amino acid sequence of the inventive proteins can also be linked to an identification peptide such as that described by Hopp et al., Bio/Technology 6:1204 (1988; FLAG™). Such a highly antigenic peptide provides an epitope reversibly bound by a specific monoclonal antibody, enabling rapid assay and facile purification of expressed recombinant protein. The sequence of Hopp et al. is also specifically cleaved by bovine mucosal enterokinase, allowing removal of the peptide from the purified protein.

Fusion proteins further comprise the amino acid sequence of a RANK linked to an immunoglobulin Fc region. An exemplary Fc region is a human IgG₁ having an amino acid sequence set forth in SEQ ID NO:3. Fragments of an Fc region may also be used, as can Fc muteins. For example, certain residues within the hinge region of an Fc region are critical for high affinity binding to FcγRI. Canfield and Morrison (J. Exp. Med. 173:1483; 1991) reported that Leu₍₂₃₄₎ and Leu₍₂₃₅₎ were critical to high affinity binding of IgG₃ to FcγRI present on U937 cells. Similar results were obtained by Lund et al. (J. Immunol. 147:2657, 1991; Molecular Immunol. 29:53, 1991). Such mutations, alone or in combination, can be made in an IgG₁ Fc region to decrease the affinity of IgG₁ for FcR. Depending on the portion of the Fc region used, a fusion protein may be expressed as a dimer, through formation of interchain disulfide bonds. If the fusion proteins are made with both heavy and light chains of an antibody, it is possible to form a protein oligomer with as many as four RANK regions.

In another embodiment, RANK proteins further comprise an oligomerizing peptide such as a zipper domain. Leucine zippers were originally identified in several DNA-binding proteins (Landschulz et al., Science 240:1759, 1988). Zipper domain is a term used to refer to a conserved peptide domain present in these (and other) proteins, which is responsible for multimerization of the proteins. The zipper domain comprises a repetitive heptad repeat, with four or five leucine, isoleucine or valine residues interspersed with other amino acids. Examples of zipper domains are those found in the yeast transcription factor GCN4 and a heat-stable DNA-binding protein found in rat liver (C/EBP; Landschulz et al., Science 243:1681, 1989). Two nuclear transforming proteins, fos and jun, also exhibit zipper domains, as does the gene product of the murine proto-oncogene, c-myc (Landschulz et al., Science 240:1759, 1988). The products of the nuclear oncogenes fos and jun comprise zipper domains that preferentially form a heterodimer (O'Shea et al., Science 245:646, 1989; Turner and Tjian, Science 243:1689, 1989). A preferred zipper moiety is that of SEQ ID NO:6 or a fragment thereof. This and other zippers are disclosed in U.S. Pat. No. 5,716,805.

Other embodiments of useful proteins include RANK polypeptides encoded by DNAs capable of hybridizing to the DNA of SEQ ID NO:1 under moderately stringent conditions (prewashing solution of 5×SSC, 0.5% SDS, 1.0 mM EDTA (pH 8.0) and hybridization conditions of 50° C., 5×SSC, overnight) to the DNA sequences encoding RANK, or more preferably under stringent conditions (for example, hybridization in 6×SSC at 63° C. overnight; washing in 3×SSC at 55° C.), and other sequences which are degenerate to those which encode the RANK. In one embodiment, RANK polypeptides are at least about 70% identical in amino acid sequence to the amino acid sequence of native RANK protein as set forth in SEQ ID NO:2 for human RANK and NO:5 for murine RANK. In a preferred embodiment, RANK polypeptides are at least about 80% identical in amino acid sequence to the native form of RANK; most preferred polypeptides are those that are at least about 90% identical to native RANK.

Percent identity may be determined using a computer program, for example, the GAP computer program described by Devereux et al. (Nucl. Acids Res. 12:387, 1984) and available from the University of Wisconsin Genetics Computer Group (UWGCG). For fragments derived from the RANK protein, the identity is calculated based on that portion of the RANK protein that is present in the fragment

The biological activity of RANK analogs or muteins can be determined by testing the ability of the analogs or muteins to bind RANKL (SEQ ID NOS:7 and 8), for example as described in the Examples herein. Suitable assays include, for example, an enzyme immunoassay or a dot blot, and assays that employ cells expressing RANKL. Suitable assays also include, for example, inhibition assays, wherein soluble RANK is used to inhibit the interaction of RANKL with membrane-bound or solid-phase associated RANK (i.e., signal transduction assays). Such methods are well known in the art.

RANKL and RANK are important factors in osteoclastogenesis. RANK is expressed on osteoclasts and interacts with RANK ligand (RANKL) to mediate the formation of osteoclast-like (OCL) multinucleated cells. This was shown by treating mouse bone marrow preparations with M-CSF (CSF-1) and soluble RANKL for 7 days in culture. No additional osteoclastogenic hormones or factors were necessary for the generation of the multinucleated cells. Neither M-CSF nor RANKL alone led to the formation of OCL. The multinucleated cells expressed tartrate resistant acid phosphatase and were positive for [¹²⁵]I-calcitonin binding. The tyrosine kinase c-src was highly expressed in multinucleated OCL and a subset of mononuclear cells as demonstrated by immunofluorescence microscopy. (See Example 2).

### Purification of Recombinant RANK

Purified RANK, and homologs or analogs thereof are prepared by culturing suitable host/vector systems to express the recombinant translation products of the DNAs of the present invention, which are then purified from culture media or cell extracts. For example, supernatants from systems which secrete recombinant protein into culture media can be first concentrated using a commercially available protein concentration filter, for example, an Amicon or Millipore Pellicon ultrafiltration unit.

Following the concentration step, the concentrate can be applied to a suitable purification matrix. For example, a suitable affinity matrix can comprise a counter structure protein or lectin or antibody molecule bound to a suitable support. Alternatively, an anion exchange resin can be employed, for example, a matrix or substrate having pendant diethylaminoethyl (DEAE) groups. The matrices can be acrylamide, agarose, dextran, cellulose or other types commonly employed in protein purification. Alternatively, a cation exchange step can be employed. Suitable cation exchangers include various insoluble matrices comprising sulfopropyl or carboxymethyl groups. Sulfopropyl groups are preferred. Gel filtration chromatography also provides a means of purifying the inventive proteins.

Affinity chromatography is a particularly preferred method of purifying RANK and homologs thereof. For example, a RANK expressed as a fusion protein comprising an immunoglobulin Fc region can be purified using Protein A or Protein G affinity chromatography. Moreover, a RANK protein comprising an oligomerizing zipper domain may be purified on a resin comprising an antibody specific to the oligomerizing zipper domain. Monoclonal antibodies against the RANK protein may also be useful in affinity chromatography purification, by utilizing methods that are well-known in the art. A ligand may also be used to prepare an affinity matrix for affinity purification of RANK.

Finally, one or more reversed-phase high performance liquid chromatography (RP-HPLC) steps employing hydrophobic RP-HPLC media, e.g., silica gel having pendant methyl or other aliphatic groups, can be employed to further purify a RANK composition. Suitable methods include those analogous to the method disclosed by Urdal et al. (J. Chromatog. 296:171, 1984). Some or all of the foregoing purification steps, in various combinations, can also be employed to provide a homogeneous recombinant protein.

Recombinant protein produced in bacterial culture is usually isolated by initial extraction from cell pellets, followed by one or more concentration, salting-out, aqueous ion exchange or size exclusion chromatography steps. Finally, high performance liquid chromatography (HPLC) can be employed for final purification steps. Microbial cells employed in expression of recombinant protein can be disrupted by any convenient method, including freeze-thaw cycling, sonication, mechanical disruption, or use of cell lysing agents. Fermentation of yeast which express the inventive protein as a secreted protein greatly simplifies purification.

Protein synthesized in recombinant culture is characterized by the presence of cell components, including proteins, in amounts and of a character which depend upon the purification steps taken to recover the inventive protein from the culture. These components ordinarily will be of yeast, prokaryotic or non-human higher eukaryotic origin and preferably are present in innocuous contaminant quantities, on the order of less than about 1 percent by weight. Further, recombinant cell culture enables the production of the inventive proteins free of other proteins which may be normally associated with the proteins as they are found in nature in the species of origin.

### Uses and Administration of RANK Compositions

The present invention provides methods of using therapeutic compositions comprising a protein and a suitable diluent and carrier. These methods involve the use of therapeutic compositions of RANK or soluble fragments of RANK for regulating an immune or inflammatory response. Further included within the present invention are methods for regulating osteoclast activity by administering therapeutic compositions of RANK or soluble RANK fragments to an individual in amounts sufficient to decrease excess bone resorption. Typically, the individual is inflicted with excess bone resorption and suffers from the effects of hypercalcemia, has symptoms of hypercalcemia, or is suffering a disease that involves excessive bone resorption. In addition to regulating osteoclast activity, the methods described herein are applicable to inhibiting osteoclast activity, regulating osteoclast generation and inhibiting osteoclast generation in individuals inflicted with excess bone resorption. In connection with the methods described herein, the present invention contemplates the use of RANK in conjunction with soluble cytokine receptors or cytokines, or other osteoclast/osteoblast regulatory molecules.

Soluble forms of RANK and other RANK antagonists such as antagonistic monoclonal antibodies can be administered for the purpose of inhibiting RANK-induced induction of NF-κB activity. NF-κB is a transcription factor that is utilized extensively by cells of the immune system, and plays a role in the inflammatory response. Thus, inhibitors of RANK signalling will be useful in treating conditions in which signalling through RANK has given rise to negative consequences, for example, toxic or septic shock, or graft-versus-host reactions. They may also be useful in interfering with the role of NF-κB in cellular transformation. Tumor cells are more responsive to radiation when their NF-κB is blocked; thus, soluble RANK (or other antagonists of RANK signalling) will be useful as an adjunct therapy for disease characterized by neoplastic cells that express RANK.

In connection with the methods described herein, RANK ligand (RANKL) on osteoblasts or stromal cells is known to interact with RANK on osteoclast progenitor surfaces signaling an event that leads to the differentiation of osteoclast precursors into osteoclasts. (See Example 2 below.) Thus, RANK, and in particular soluble forms of RANK, is useful for the inhibition of the RANKL-mediated signal transduction that leads to the differentiation of osteoclast precursors into osteoclasts. Soluble forms of RANK are also useful for the regulation and inhibition of osteoclast activity, e.g. bone resorption. By interfering with osteoclast differentiation, soluble forms of RANK are useful in the amelioration of the effects of osteoclastogenesis in disease conditions in which there is excess bone break down. Such disease conditions include Paget's disease, osteoporosis, and cancer. Many cancers metastasize to bone and induce bone breakdown by locally disrupting normal bone remodeling. Such cancers can be associated with enhanced numbers of osteoclasts and enhanced amount of osteoclastic bone resorption resulting in hypercalcemia. These cancers include, but are not limited to, breast cancer, multiple myeloma, melanomas, lung cancer, prostrate, hematologic, head and neck, and renal. (See Guise et al. Endocrine Reviews, 19(1):18-54, 1998.) Soluble forms of RANK can be administered to such cancer patients to disrupt the osteoclast differentiation pathway and result in fewer numbers of osteoclast, less bone resorption, and relief from the negative effects of hypercalcemia.

Other cancers do not metastasize to bone, but are known to act systemically on bone to disrupt bone remodeling and result in hypercalcemia. (See Guise et al. Endocrine Reviews, 19(1):18-54, 1998.) In accordance with this invention, RANKL has been found on the surface of certain squamous cells that do not metastasize to bone but are associated with hypercalcemia. (See Example 3 below) Squamous cells that are associated with hypercalcemia also express M-CSF (CSF-1), a cytokine that, together with RANKL, stimulates the proliferation and differentiation of osteoclast precursors to osteoclasts. In accordance with the present invention, it has been discovered that M-CSF directly upregulates RANK on surfaces of osteoclast precursors. When squamous cells release excessive amounts of CSF-1, increased expression of RANK occurs on the surfaces of osteoclast precursors. Thus, there is a higher probability that RANK will interact with RANKL on osteoblasts or stromal cells to produce increased numbers of osteoclasts, resulting in an enhanced amount of bone break down and hypercalcemia.

In addition to the ameliorating the effects of cancers that metastasize to bone, the present invention provides methods for ameliorating the systemic effects, e.g. hypercalcemia, of cancers that are associated with excess osteoclast activity (e.g. squamous cell carcinomas). Such methods include administering soluble forms of RANK in amounts sufficient to interfere with the RANK/RANKL signal transduction that leads to the differentiation of osteoclast precursors into osteoclasts. Fewer osteoclasts lead to reduced bone resorption and relief from the negative effects of hypercalcemia.

For therapeutic use, purified protein is administered to an individual, preferably a human, for treatment in a manner appropriate to the indication. Thus, for example, RANK protein compositions administered to regulate osteoclast function can be given by bolus injection, continuous infusion, sustained release from implants, or other suitable technique. Typically, a therapeutic agent will be administered in the form of a composition comprising purified RANK, in conjunction with physiologically acceptable carriers, excipients or diluents. Such carriers will be nontoxic to recipients at the dosages and concentrations employed.

Ordinarily, the preparation of such protein compositions entails combining the inventive protein with buffers, antioxidants such as ascorbic acid, low molecular weight (less than about 10 residues) polypeptides, proteins, amino acids, carbohydrates including glucose, sucrose or dextrins, chelating agents such as EDTA, glutathione and other stabilizers and excipients. Neutral buffered saline or saline mixed with conspecific serum albumin are exemplary appropriate diluents. Preferably, product is formulated as a lyophilizate using appropriate excipient solutions (e.g., sucrose) as diluents. Appropriate dosages can be determined in trials. The amount and frequency of administration will depend, of course, on such factors as the nature and severity of the indication being treated, the desired response, the condition of the patient, and so forth.

Soluble forms of RANK and other RANK antagonists such as antagonistic monoclonal antibodies can be administered for the purpose of inhibiting RANK-induced osteoclastogenesis. It is desirable to inhibit osteoclastogenesis in various disease states in which excess bone loss occurs. Examples include osteoporosis, Pagett's disease, and various cancers. Various animal models of these diseases are known in the art; accordingly, it is a matter of routine experimentation to determine optimal dosages and routes of administration of soluble RANK, first in an animal model and then in human clinical trials.

The following examples are offered by way of illustration, and not by way of limitation. Those skilled in the art will recognize that variations of the invention embodied in the examples can be made, especially in light of the teachings of the various references cited herein, the disclosures of which are incorporated by reference.

### Example 1

This example describes a plate binding assay useful in comparing the ability of various ligands to bind receptors. The assay is performed essentially as described in Smith et al., Virology 236:316 (1997). Briefly, 96-well microtiter plates are coated with an antibody to human Fc (i.e., polyclonal goat anti human Fc). Receptor/Fc fusion proteins are then added, and after incubation, the plates are washed. Serial dilutions of the ligands are then added. The ligands may be directly labeled (i.e., with ¹²⁵I), or a detecting reagent that is radioactively labeled may be used. After incubation, the plates are washed, specifically bound ligands are released, and the amount of ligand bound quantified.

Using this method, RANK/Fc and OPG/Fc were bound to 96-well plates. In an indirect method, a RANKL/zipper fusion is detected using a labeled antibody to the zipper moiety. It was found that human OPG/Fc binds mRANKL at 0.05 nM, and human RANK/Fc binds mRANKL at 0.1 nM. These values indicate similar binding affinities of OPG and RANK for RANKL, confirming the utility of RANK as an inhibitor of osteoclast activity in a manner similar to OPG.

### Example 2

The following describes the formation of osteoclast like cells from bone marrow cell cultures using a soluble RANKL in the form of soluble RANKL/leucine zipper fusion protein (RANKL LZ).

Using RANKL LZ at 1 μg/ml, osteoclasts were generated from murine bone marrow (BM) in the presence of CSF-1. These osteoclasts are formed by the fusion of macrophage-like cells and are characterized by their TRAP (tartrate-resistant acid phosphatase) positivity. No TRAP⁺ cells were seen in cultures containing CSF-1 alone or in cultures containing CSF-1 and TRAIL LZ (a control for the soluble RANKL LZ). Even though human and monkey bone marrow contains more contaminating fibroblasts than murine bone marrow, osteoclasts were generated from murine and monkey bone marrow with the combination of CSF-1 and soluble RANKL LZ. In a dose-response study using murine bone marrow and suboptimal amounts of CSF-1 (40 ng/ml), the effects of soluble RANKL LZ plateaued at about 100 ng/ml.

The effect of soluble RANKL LZ on proliferation of cells was studied in the same cultures using Alamar Blue. After 5 days, the proliferative response was lower in cultures containing CSF-1 and RANKL LZ than in those containing CSF-1 alone. The supports the observation that soluble RANKL LZ is inducing osteoclast differentiation. When CSF-1 and RANKL LZ are washed out of murine BM cultures at day 7 or 8, cells do not survive if they are recultured in medium or in RANKL LZ alone. In contrast, cells do survive if recultured in CSF-1. When RANKL LZ was added to these cultures there was no added benefit. Thus, the combination of CSF-1 and RANKL are required for the generation of osteoclast. Additionally, once formed, CSF-1 is sufficient to maintain their survival in culture.

Finally, using human bone marrow, soluble anti-human RANK mAb and immobilized anti-human RANK mAb were compared to RANKL LZ for the generation of osteoclasts in the presence of CSF-1. Immobilized M331 and RANKL LZ were found to be equally effective for osteoclast generation while soluble M331 was superior to both immobilized antibody and RANKL LZ. This confirms that the osteoclast differentiating activity of RANKL is mediated through RANK rather than via an alternative receptor.

Since osteoclasts cannot readily be harvested and analyzed by flow cytometry, ¹²⁵I-labeled calcitonin binding assays were used to identify osteoclasts (the calcitonin receptor is considered to be an osteoclast-specific marker). Osteoclasts generated from murine BM cultured with CSF-1 and RANKL LZ for 9 days showed binding of radiolabeled calcitonin confirming their osteoclast identity.

### Example 3

In order to determine RANKL expression by either of two different squamous cell carcinomas, standard Western blot and RT-PCR studies were performed on MH-85 and OKK cells. One of these carcinoma cells, the MH-85 cells, is associated with hypercalcemia.

The results confirmed that MH-85 and OKK squamous cells express RANKL. MH-85 cells, in addition to being linked with hypercalcemia in patients inflicted with this carcinoma, also express M-CSF (CSF-1). It was also determined that CSF-1 upregulates RANK expression on osteoclast precursors. The enhanced amount of CSF-1 in MH-85 type squamous cell cancer patients can lead to an upregulation of RANK and increased RANK interaction with RANKL. Signals transduced by RANK and RANKL interaction result in increased numbers of mature osteoclasts and bone breakdown. Since soluble forms of RANK can inhibit the RANK/RANKL interaction, administering a soluble form of RANK (e.g. the extracellular region of RANK fused to an Fc) to a squamous cell cancer patient provides relief from adverse effects of this cancer, including hypercalcemia.

We claim:

1. A method of regulating osteoclast activity, the method comprising causing a soluble RANK to bind RANKL.

2. The method of claim 1, wherein the soluble RANK is encoded by a DNA selected from the group consisting of:
(a) a DNA encoding a protein having an amino acid sequence as set forth in SEQ ID NO:2, wherein the protein has an amino terminus selected from the group consisting of an amino acid between amino acid 1 and amino acid 33, inclusive, of SEQ ID NO:62, and a carboxy terminus selected from the group consisting an amino acid between amino acid 196 and amino acid 616, inclusive;
(b) a DNA encoding a protein having an amino acid sequence as set forth in SEQ ID NO:6, wherein the protein has an amino terminus selected from the group consisting of an amino acid between amino acid 1 and amino acid 30, inclusive, of SEQ ID NO:6, and a carboxy terminus selected from the group consisting an amino acid between amino acid 197 and amino acid 625, inclusive;
(c) DNA molecules capable of hybridization to the DNA of (a) or (b) under stringent conditions, and which encode RANK polypeptides that bind RANKL; and
(d) DNA molecules encoding fragments of proteins encoded by the DNA of (a), (b) or (c), wherein the fragments of RANK polypeptides bind RANKL.

3. The method of claim 2, wherein the RANK is at least about 80% identical in amino acid sequence to native RANK

4. The method of claim 3, wherein the RANK further comprises a polypeptide selected from the group consisting of an immunoglobulin Fc domain, an immunoglobulin Fc mutein, a FLAG™ tag, a peptide comprising at least about 6 His residues, a leucine zipper, and combinations thereof.

5. A method of ameliorating effects of excess bone loss, comprising administering a soluble RANK polypeptide composition to an individual at risk for excess bone loss, and allowing the soluble RANK to bind RANKL and inhibit binding thereof to cells expressing RANK.

6. The method of claim 5, wherein the individual is at risk from or suffers from a condition selected from the group consisting of osteoporosis, Pagett's disease, and bone cancer, and cancers associated with hypercalcemia.

7. The method of claim 5, wherein the soluble RANK is encoded by a DNA selected from the group consisting of:
(a) a DNA encoding a protein having an amino acid sequence as set forth in SEQ ID NO:2, wherein the protein has an amino terminus selected from the group consisting of an amino acid between amino acid 1 and amino acid 33, inclusive, of SEQ ID NO:62, and a carboxy terminus selected from the group consisting an amino acid between amino acid 196 and amino acid 616, inclusive;
(b) a DNA encoding a protein having an amino acid sequence as set forth in SEQ ID NO:6, wherein the protein has an amino terminus selected from the group consisting of an amino acid between amino acid 1 and amino acid 30, inclusive, of SEQ ID NO:6, and a carboxy terminus selected from the group consisting an amino acid between amino acid 197 and amino acid 625, inclusive;
(c) DNA molecules capable of hybridization to the DNA of (a) or (b) under stringent conditions, and which encode RANK polypeptides that bind RANKL; and
(d) DNA molecules encoding fragments of proteins encoded by the DNA of (a), (b) or (c), wherein the fragments of RANK polypeptides bind RANKL.

8. The method of claim 7, wherein the RANK is at least about 80% identical in amino acid sequence to native RANK

9. The method of claim 8, wherein the RANK further comprises a polypeptide selected from the group consisting of an immunoglobulin Fc domain, an immunoglobulin Fc mutein, a FLAG™ tag, a peptide comprising at least about 6 His residues, a leucine zipper, and combinations thereof.

10. The method of claim 6, wherein the soluble RANK is encoded by a DNA selected from the group consisting of:
(a) a DNA encoding a protein having an amino acid sequence as set forth in SEQ ID NO:2, wherein the protein has an amino terminus selected from the group consisting of an amino acid between amino acid 1 and amino acid 33, inclusive, of SEQ ID NO:62, and a carboxy terminus selected from the group consisting an amino acid between amino acid 196 and amino acid 616, inclusive;
(b) a DNA encoding a protein having an amino acid sequence as set forth in SEQ ID NO:6, wherein the protein has an amino terminus selected from the group consisting of an amino acid between amino acid 1 and amino acid 30, inclusive, of SEQ ID NO:6, and a carboxy terminus selected from the group consisting an amino acid between amino acid 197 and amino acid 625, inclusive;
(c) DNA molecules capable of hybridization to the DNA of (a) or (b) under stringent conditions, and which encode RANK polypeptides that bind RANKL; and
(d) DNA molecules encoding fragments of proteins encoded by the DNA of (a), (b) or (c), wherein the fragments of RANK polypeptides bind RANKL.

11. The method of claim 10, wherein the RANK is at least about 80% identical in amino acid sequence to native RANK

12. The method of claim 11, wherein the RANK further comprises a polypeptide selected from the group consisting of an immunoglobulin Fc domain, an immunoglobulin Fc mutein, a FLAG™ tag, a peptide comprising at least about 6 His residues, a leucine zipper, and combinations thereof.
